(19) United States
(12) Patent Application Publication
(10) Pub. No.: US 2020/0168344 A1
(43) Pub. Date: May 28, 2020

(54) PHYSICIAN COMMUNICATION SYSTEM

(71) Applicant: Donald L Dixon, Chapel Hill, NC (US)

(72) Inventor: Donald L Dixon, Chapel Hill, NC (US)

(21) Appl. No.: 16/199,519

(22) Filed: Nov. 26, 2018

(51) Int. Cl. G16H 80/00; G16H 20/00; A61F 2/30

(52) U.S. Cl. CPC G16H 80/00; A61F 2/30; G16H 20/00

(57) ABSTRACT

This invention discloses a method for selection of a proper prosthetic device for a patient utilizing joint decision making and information gathering by a patient, clinician, and physician utilizing a computerized Physician Communication System, wherein a clinician evaluates a patient and enters the patient's information into a smart device that communicates with a computer system and a dynamic database that stores the information and also has prosthetic device specific information where software is capable of propagating the information through other devices, a computer system and the database in real time where the clinician selects a prosthetic device for a patient, uploads the selection, and a physician consults the database to evaluate the choice of device for the patient, and can accept, change, or otherwise edit the information and choice based on the physician's examination of the patient.

### CROSS-REFERENCE TO RELATED APPLICATIONS

Applicant is claiming the benefit of a prior-filed provisional application, Application No. 62/592,648 filed Nov. 30, 2017, under 35 U.S.C. 119(e), and states that the nonprovisional application was filed not later than 12 months after the date on which the provisional application was filed, and that the inventor is the same and the proper filing fee for the provisional application was paid.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not Applicable

### STATEMENT REGARDING PRIOR DISCLOSURES BY THE INVENTOR OR A JOINT INVENTOR

Not Applicable

### BACKGROUND OF THE INVENTION

### Field of the Invention

This invention generally relates to the field of medical devices, particularly prosthetic devices. More particularly, the present invention relates to a method of selection of the proper prosthetic to recommend to a particular patient in need of such a device. Further, this invention relates to a method to simplify the selection of a specific prosthetic device and facilitate a physician's choice of prosthetic for a particular patient taking into account several determining variables. Still further, the present invention relates to a method and system that allows a prosthetist to store findings, recommendations, and treatment notes on a smart device or computer, and a physician to retrieve, review and edit the information. Further, the invention relates to a method by which selection of a specific prosthetic device will initiate a computer to populate detailed schedules with appropriate L-codes and other information, and generate a letter to physicians simply by selection of the specific prosthetic device. The present invention utilizes a dynamic data base to store information gathered from various sources and centrally stored for retrieval from any number of smart devices or computers. Thus, the present invention relates to a method and system in which a prosthetist utilizes specialized knowledge to interview a patient and select a proper prosthesis for the patient's use. The invention facilitates and automates the selection process by utilizing predetermined characteristics and information sorted into the database and linked to individual codes or characteristics of devices. The invention still further allows a physician to review the selection of a particular prosthetic for a specific patient and either accept the diagnosis or edit and revise the diagnosis leading to the selection of an alternate device. Still further, the invention provides a centralized, internet based, dynamic, data base that allows downloading and uploading of information through the use of a smart device. Selecting the proper prosthetic can be time consuming, difficult, and lead to inconsistent results between practitioners. Additionally, the knowledge of the proper L-Codes for specific features of devices is difficult to acquire, and many physicians do not have time to learn the L-Codes. Thus a method to identify the proper L-Codes applicable to the selected prosthetic device is needed. The present invention overcomes these issues.

Medical care and treatment options today typically involve multiple parties. The arrangement of one patient to one physician is all but gone. Today's healthcare system consists of multiple parties involved in the treatment of a patient. Often times there are multiple physicians, general practitioners and specialists, nurse practitioners, physician's assistants, DMs, residents, and other medical personnel involved with the treatment of a single patient. In addition to medical personnel, there can be insurance companies, HMOs, unions, health savings plan administrators, and other third-parties involved in the treatment and/or the financial aspects of a patient's care. Moreover, Medicare and Medicaid are involved in patient care in an increasing rate, and to allow Medicare to pay for certain treatment often requires compliance with many rules and regulations. Often times these professionals are in different offices, sometimes in different geographic locations, or even across the country. A need exists to coordinate the efforts of these individuals and institutions to streamline the patient's treatment, ensure that the patient's needs are being met, and reduce the costs of the treatment whenever possible.

Today's patients are typically more informed about their treatment and seek to be more involved in the choice of their treatment. Thanks to the proliferation of medical information over the Internet and through various media, such as television, patients are often well informed about their particular medical needs and the options available for treatment. Patients typically appreciate being informed and participating in the decision making process concerning their treatment. However, most patients do not have the expert training possessed by the healthcare professionals. Thus, a need exists for a system that will allow for input by the patient in determining what treatment methods are recommended and followed by the healthcare professional.

This is particularly true in cases where the patient has lost a limb and is in need of a prosthetic device. Advances in technology have resulted in the proliferation of a huge number of prosthetic devices available to patients. Indeed, medical professionals can devote their entire practice to simply the selection of the proper prosthetic for a particular patient. This medical professional is known as a Prosthetist, and this individual has the knowledge of the various devices available for use by patients. In the majority of cases, the prosthetist has superior knowledge to that of physicians regarding the proper device for use by a particular patient. This is in part due to the acquired knowledge gained by the prosthetist over the course of their training and career. A typical physician simply does not have time to maintain proficiency in the field of prosthetics in addition to the field of medicine. A prosthetist however, can devote time to a mastery of the knowledge of the available prosthetic devices available to patients. A prosthetist can work for a particular clinic devoted to the selection, fit, and adjustment of prosthetic devices for patients. Such persons are referred to as a “clinician.”

In the case of a patient requiring a prosthetic, care must be taken in selecting which prosthetic is appropriate for the patient. A patient must self-evaluate to determine such things as the need for reduction of weight, the ability to change components, or other subjective criteria. Allowing the patient to discuss such needs with a healthcare professional, particularly a clinician, allows the gathering of data important to the selection of the proper prosthetic. Further, it involves the patient in the decision making process which helps with the psychological aspects of adjusting to the particular prosthetic device.

In addition to patients gaining further knowledge from electronic sources, healthcare professionals also have access to vast amounts of data from which they can consult before forming their opinions and diagnoses. Further, the various levels of training that different healthcare professionals have received can put them at different levels when considering treatment options for a specific patient. Their differing backgrounds combined with the vast array of information available for research leads to inconsistent conclusions, and sometimes can lead to inconsistent treatment decisions. Thus, a need exists for a centralized information or data source specific to a particular patient. This will allow the various healthcare professionals to see what each is doing and follow such treatment or alter it given specific criteria. This is especially true when a particular patient moves from one healthcare provider to another. As the general population becomes increasingly mobile, it is highly likely that patients will end up seeing multiple healthcare professionals, sometimes separated by large geographical distances. Thus, a need exists for a method to coordinate the treatment plan and history of a patient such that various health care professionals can participate or at least have access to this patient information.

Third parties involved in the health care system, in particularly the entities involved in the financial aspects of treatment, also need a means to be involved in the financial aspect of the treatment of the patient. These entities are precluded from participating in selecting the appropriate treatment due to the conflict of interest, but they have an interest in a streamlined, efficient, error-free means of selecting the appropriate treatment for a patient because it lowers the overall costs of treatment. This is especially true in cases where Medicare or Medicaid is responsible for part or all of the expense of the treatment. In situations where a prosthetic is necessary for the treatment of the patient, Medicare has assigned specific codes “L-Codes” to particular functions that prosthetics are able to perform or handle in order to aid in the selection and treatment of patients, and streamline the process of Medicare paying for such devices.

The particular L-code determined for a function of the device is a standard set by Medicare. The list of L-codes applicable to the prosthetic industry is long and unfamiliar to most medical practitioners. Thus, it is likely that only a skilled prosthetist will have familiarity with the L-codes used by the medicare system. Thus, a method to easily associate required characteristics of a prosthetic device required by a patient by L-code is needed. Further, as Medicare is a government function, L-codes are likely to change over time as new legislation and regulations are propagated. Thus, a need exists for an easy way to propagate the changes enacted by Medicare to the healthcare professionals in a timely manner. Further, a need exists to be able to quickly and accurately determine the proper L-Code for each aspect of a prosthetic selected for a particular patient. The present invention is able to adapt to update the list of L-codes in order to respond to changes by Medicare, and it is able to list all applicable L-Codes for a specific prosthetic device upon selection of that device by a clinician.

In a typical situation where a patient is in need of a prosthetic device, he or she will go to see the primary care physician, or a specialist in orthopedics or other area where prosthetic devices are utilized. However, the physician typically will not have the desired familiarity with the vast array of prosthetic devices available in the market. As a result, the physician will typically refer a patient to a prosthesis clinic for an evaluation and recommendation on what type of device is needed. Until the present invention, there has been no standard communication system available to physicians that will allow a standardized communication procedure between the physician and the prosthetist or clinician. These professionals are left relying on methods of communications that are antiquated, slow, and at times unreliable. This can lead to delay and frustration on the part of the patient who often times is grappling with the emotional turmoil associated with losing a limb. Thus, a need exists to overcome these issues with the prior art; the present invention overcomes these issues and provides a solution to these problems.

### BRIEF SUMMARY OF THE INVENTION

One of the purposes of the present invention is to provide a method for selection of a proper prosthetic device for a patient utilizing joint decision making and information gathering by a patient, clinician, and physician. The present invention also provides for the use of a computerized Physician Communication System, that allows communication through digital means between the clinician, the patient, and the physician. The method generally is performed by having a Physician Computer System (PCS) that is operated and controlled by the use of a smart device. The smart device communicates with a computer system storing a dynamic database. The database contains patient specific information and prosthetic device specific information, including L-Codes, that is entered into the computer database by clinician staff, physician staff, the patient, or other designated individuals responsible for gathering a patient's basic information. In the case of the L-Codes, these are assigned by the Government as part of the Medicare program and are subject to legislative change.

The invention has specific software loaded on the various smart devices to operate the PCS. The software can be downloaded via the Internet from the server, and can be adapted to be an application capable of running on any smart device such as a tablet, laptop, or cell phone. The software allows one to enter prosthetic device information and patient information that is gathered from interviewing a patient, in the case of patient information, or that is obtained from a manufacturer or other source, in the case of the prosthetic device information. Further, the present invention contemplates that the software is capable of uploading and downloading the information to and from the database in real time.

The present invention further includes having a clinician evaluate a patient to gather information from the patient, and enter the patient's information on the smart device. The information would include the clinician's selection of a prosthetic device for treatment of the patient based upon the clinician's evaluation of the patient and familiarity with the various prosthetic devices available to treat the particular needs and desires of the patient. The software then will upload the clinician's selection of a specific prosthetic device to the PCS database through the Internet, a USB connection, or another acceptable method of moving the information between devices. The software will also upload all the appropriate L-Codes that are applicable to the specific functions or parts of the selected prosthetic.

Once the clinician has made a selection of a particular prosthetic device and uploaded the details to the PCS database, the physician becomes involved in the process. The physician will meet with the patient and will consult the PCS database. The physician uses a smart device to review the information from the PCS system pertaining to the particular patient. The physician will evaluate the patient for the physical and mental factors necessary for using the specific prosthetic device selected by the clinician and entered into the PCS. The physician verifies that the physical and mental requirements for the chosen prosthetic device are met by the patient. If they are, the physician indicates on the smart device that the physician accepts the chosen prosthetic device selected by the clinician by confirming on the smart device its selection. The physician will typically enter a verification of the selected prosthetic device into the PCS database through the smart device.

The present invention further contemplates a PCS wherein the smart device is in communication with a computer system storing a dynamic database where the information in the database is capable of being updated and manipulated in real time. The smart device can use the installed software to selectively download and upload data to main PCS database according to parameters selected by a user. For example, the software can be configured to download only new patient files entered in a specific time period. Or, the device can be configured to download all files from a particular date range. As one who is skilled in the art can appreciate, there are multiple ways to make use of the information, all of which are contemplated as part of the instant invention.

It is a further object of the present invention to provide a method in which the selection and verification of a particular prosthetic device for a patient is dependent upon various factors related to the device and the patient. Such factors that would be entered into the PCS and would include such information as a patient's particular condition, goals, activities in which the patient desires to participate, treatment options, costs, risks, and physical requirements. It is further contemplated that the information included in the PCS would include device specific information such as K codes, corresponding L-codes, any particular requirements for the specific device, and other device specific information necessary for the treatment of the patient. Also, if there were other data or information necessary for the selection and treatment of a patient, this information would be included in the PCS database.

The present invention further contemplates that the software would store the information specific to a prosthetic device and organize it into dynamic checkboxes. Thus, the clinician and physician could simply touch the checkbox to indicate that such information is applicable to the particular patient. This would contribute to saving the clinician and physician time when meeting with the patient since there would be less time spent entering information into the smart device.

Another object of the present invention is to provide a method wherein one enters patient information into a smart device or computer installed with software capable of receiving, storing, and transmitting the information to a database where the database is capable of receiving information from multiple smart devices at the same time. Thus, multiple clinicians and multiple physicians can be using the database at the same time without interfering with each other's use. Moreover, it is also contemplated that the database can be accessible to other devices through the Internet, such as computers, copiers, etc.

The method further contemplates sending a patient to a clinician for an initial recommendation of a prosthetic device. When the patient first meets with the clinician, the clinician enters patient information and or prosthetic device information into a smart device or computer that transmits the information to the PCS database. The patient then goes to see a physician who downloads the patient's file from the PCS database and meets with the patient. The physician meets with and evaluates the patient and the initially recommended prosthetic device for compatibility. Once the physician has made a determination of whether the selected device and the patient are compatible, the physician will verify the initial recommended prosthetic device through said smart device. The smart device will then transmit the verification to the PCS database.

The present invention also contemplates a method for selecting and prosthetic device for a patient wherein a physician determines that the initially recommended prosthetic device is not appropriate for the patient. In this case the physician would reject the clinician's initial recommended prosthetic device. This could be done by any conventional selection/deselection method, for example by deselection of a check box on the smart device. The method contemplates the physician being able to enter information on the smart device as to why the selected device and patient are incompatible. Then, the physician will evaluate the patient further for specific information that would facilitate selecting an alternate prosthetic device for the patient based upon the requirements of the patient and device and the evaluation of the physician. The invention contemplates the physician then inputting alternate recommendations or requirements for the patient into the smart device. These can be entered as necessary attributes, particular L-Codes, or other information that the physician believes is necessary for the selection of a proper prosthetic. The smart device then transmits the alternate recommendation or requirements to the PCS database for storage and retrieval. This allows the physician, clinician, and patient to have an ongoing dialogue regarding the proper prosthetic device with which to treat the patient.

It is another object of the present invention to provide an automatic conversion of information entered by a clinician into information more pertinent to a treating physician. For example, a clinician may enter a particular L-code associated with a device or patient. The software would automatically convert the entered code into information necessary for the physician to evaluate the patient to ensure the patient meets the criteria for use of the specific device. Or vice-versa, the clinician would enter a specific prosthetic device that is recommend for a patient, and the software would populate the fields with the proper L-Codes, descriptions for the particular components, K-codes, and other information specific to the prosthetic selected. This would facilitate the physician's evaluation of the patient with respect to the selected prosthetic, but would not require the physician to be familiar with the specific meanings of each code entered by the clinician.

### BRIEF DESCRIPTION OF THE SEVERAL VIEWS OF THE DRAWING(S)

FIG. 1 is a flow chart diagram illustrating the preferred embodiment of process of the claimed method.

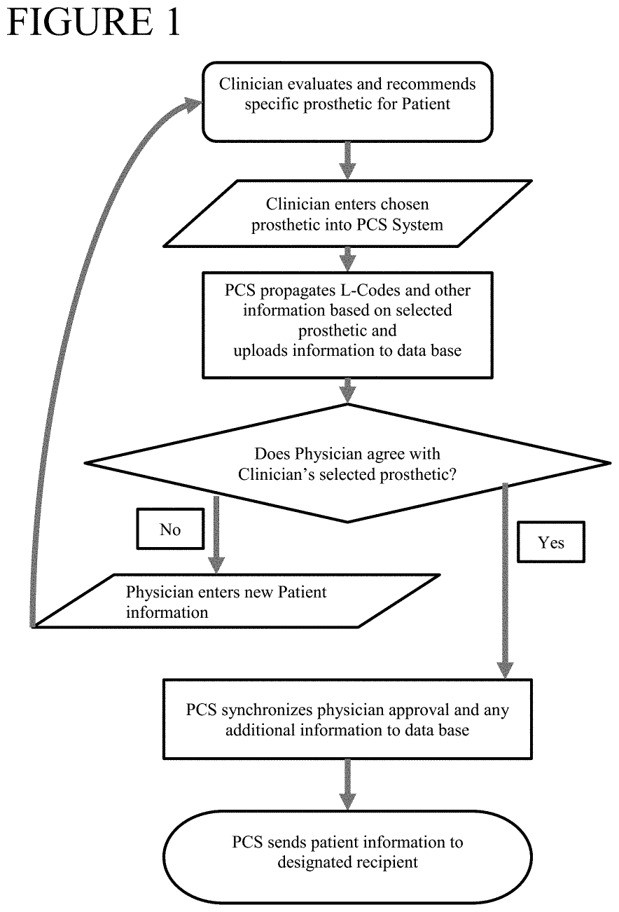

### DETAILED DESCRIPTION OF THE INVENTION

The present invention is a method to facilitate the selecting and prescribing of a particular prosthetic device to a patient by a physician, with specific input parameters provided to a prosthetic clinician to select an appropriate prosthetic device from the available devices. In a preferred embodiment, the present invention is used to facilitate the selection of a prosthetic device for a new patient who has never had a prosthetic device. While this is the preferred embodiment and will be fully described, it should be appreciated that the same method can be used when a patient wants to change devices or wants to add an additional device to one already owned. The present invention allows for modification of the internal steps to achieve these goals, and it can further be modified to achieve various other goals without departing from the spirit of the invention.

At the outset, a selection of the specific prosthetic device will be done by the clinician. The “clinician” is an individual trained in prosthetic devices, a prosthetist. The clinician has specialized training and knowledge relative to the devices available to patients and is also familiar with the rules, regulations, and other requirements of Medicare, Medicaid, and other governmental programs that may cover part of the cost of the prosthetic device to the patient. Further, the clinician is familiar with the L-codes as set by Medicare and can assist the other medical personnel and the patient in navigating the forms required for the governmental entities.

At its core, the present invention includes the use of a dynamic, template, software program capable of being run on a computer, tablet, phone, or other smart device. The software includes a database capability where the data generated at the various stages is kept. The database is also capable of downloading and uploading the data to the various smart devices through the use of the software program itself, or an associated program loaded on the smart device, typically referred to as an application or an “app.” Further, the present invention has the ability to upload and download the data in real time, so that the persons using the program can have the most current information available, and if anything changes, the changes will be propagated through the data base to each smart device being used.

The present invention may be more fully understood by a study of the following descriptions and steps involved in the method taught by the present invention. It also is helpful to refer to the drawings that are illustrative of the varying steps included in the invention. Where possible, the following method is described for a person first receiving a prosthetic for the first time. It should be appreciated by those skilled in the art that the present invention is not limited by the following enabling disclosure, rather the following is the contemplated best mode and preferred embodiment of the invention.

At the outset, the present invention is a dynamic database populated with predetermined descriptions, codes, and other particular information as decided by the user. For purposes of this detailed description, the present invention will be referred to as the Physician Communication System. A computer will be used as the database host, and the entities who desire to participate may link to the computer database through a smart device with Internet access. At the beginning of each workday, or whenever the system or software is to be used, or at a preset time, such as late at night or early in the morning, the Physician Communication System will update its records. In a preferred embodiment, the Physician Communication System will perform a search to locate any records that have been queued for uploading, and it will proceed to upload and assimilate those records into the database. Once this is completed, the Physician Communication System will be ready for use.

The first step in a new patient receiving a prosthetic device will include the patient meeting with a clinician skilled in what specific devices are available. The clinician will bring a smart device, preferably a tablet, into the meeting with the new patient. The clinician starts the Physician Communication System app on the smart device and all records and data in the main data base pertaining to the devices that have been updated since the app was last started on that particular smart device are downloaded into the smart device. This ensures that the data and software in the smart device is the most current and up to date. Also, the data for each patient the clinician is treating will download, thus ensuring that the data for each patient matches the data in the main database. This ensures that the Clinician has the most current information on each individual patient and each device in the data base on the smart device for ready access. Further, any changes in regulations such as L-Code assignments can be downloaded and applied to the database.

As with any medical practice, when the new patient is going to meet with the clinician for treatment and selection of a new prosthetic, certain information regarding the patient must be gathered. This is usually accomplished by the new patient filling out numerous forms with basic information such as name, address, medical insurance information, etc. Also, often times this information gathering will collect information regarding the patient's medical history and current symptoms. This is a routine administrative task and can be accomplished by office staff, or a person specifically designated to gather this information. In a preferred embodiment, the office staff will collect this basic new patient information.

When the office staff meets with the new patient prior to the new patient meeting with the clinician, the staff will collect the basic information from the new patient. Staff will interview the new patient and create a new patient profile/file in a smart device loaded with the Physician Communication System app. Staff will interview the new patient and enter the appropriate data into the smart device creating a specific patient profile or file, complete with the basic information about the patient. Once the information is entered and the patient's file is created in the Physician Communication System data base on the smart device, the office staff can select an option in the software to upload the new file to the main database. Once this is completed, any smart device with the Physician Communication System app can download the new patient's file. Thus, when the clinician meets with the new patient, the clinician can use a different smart device than that used by the staff. This allows multiple smart devices to be used simultaneously and ensures that the most current information is available.

The Clinician will meet with the new patient and enter the new patient's name into the smart device. The Physician Communication System will search for and retrieve the file for the new patient. Once the Physician Communication System locates the file, it will populate the fields in the software with the data specific to the new patient. It should also be appreciated by those skilled in the art that the Physician Communication System is capable of creating multiple files per patient, for example when a patient has more than one prosthetic. Thus, the Physician Communication System is adaptable and may be configured to accept multiple search criteria in addition to a patient's name. For example, the Physician Communication System may be configured to search for specific devices, or specific limbs. Moreover, the Physician Communication System may be configured to search for multiple entries, such as name and limb, or name and device. While the preferred embodiment disclosed and taught will use the name as the search basis, the present invention included additional capability.

Once the clinician retrieves the patient's file on the smart device, the clinician can conduct the interview. During the interview, the clinician interviews the patient and determines what the patient's goals and expectations for a prosthetic device are, including any necessary characteristics that the device must possess. This includes an evaluation of any activities in which the patient desires to participate. Further, if there are specific goals that the patient is attempting to achieve with the prosthetic device, the clinician will consider those goals in selecting the proper device to recommend.

During the interview with the new patient, the Clinician determines the “K Level” of the particular patient. The K level is typically an assigned level based on the new patient's ability to use the prosthetic for varying levels of activity. For example, a K−1 level would indicate that the patient has the potential to use a prosthesis for transfers or ambulation on level surfaces at a fixed cadence. At the other end, a K−4 level would indicate that the patient has the potential for prosthetic ambulation that exceeds basic ambulation skills, exhibiting high impact, stress, or energy levels. These would be activity levels typical of a child, active adult, or athlete.

During the interview, the clinician collects and enters information pertaining to the new patient into the smart device. It is contemplated that the entering of data is accomplished by entering the information in either a text field or, in a preferred embodiment, a series of check boxes that align to various activities or characteristics of prosthetic devices. For example, the check boxes could align to K codes. As part of the clinician's evaluation of the new patient, the clinician evaluates the new patient for various physical factors such as strength and mobility, and may evaluate the new patient for mental acquity as well. The clinician makes a determination of necessary components for the prosthetic device to have, and/or determines any specific design necessary for the prosthetic device to have in order for the patient to have the best ability to engage in the desired activity.

Based on the clinician's evaluation of the new patient, the clinician makes a determination of which prosthetic device would be best for the new patient. This decision is based upon considering how a specific device will allow the patient to achieve the goals identified in the interview, and considering which device would be most compatible with the new patient based on the clinician's evaluation of the patient. Further, if there are any specific considerations that do not fall into one of the predetermined categories, the clinician can enter these factors and considerations in a comment section of the Physician Communication System app.

Once the clinician has made a decision regarding the specific prosthetic device to recommend to the new patient, the clinician enters the selected prosthetic into the smart device prosthetic selection field. The Physician Communication System recognizes the specific prosthetic selected and automatically populates several dialog boxes preprogrammed into the Physician Communication System software. These preprogrammed fields would include L-codes, description, reasons for using, clinician recommendation, pertinent notes, and others, depending on the desired fields. The preselected data is based upon the device selected, and the information specific to the particular device is preprogrammed into the software. Thus, once the clinician enters the selected prosthetic device into the Physician Communication System, the Physician Communication System retrieves the data specific to that device and populates the fields with the prosthetic specific data, including L-Codes and the like.

When the various data fields in the software app are filled, the clinician reviews the data in the app on the smart device and makes a determination of whether the data is accurate and sufficient. If the clinician believes that the information is not sufficient, the clinician can selectively edit any of the populated fields. Thus, the Physician Communication System is designed and configured to allow the clinician to selectively edit any field that the clinician believes needs further explanation or comment. After the clinician reviews the changes, or when the clinician believes all necessary information is included in the app, the clinician will discuss the findings and the recommended prosthetic device with the patient, confirming with the patient that the selected prosthetic is acceptable to the new patient. After confirming with the patient that the selected prosthetic is acceptable, and that the information contained in the various fields in the Physician Communication System app is correct and complete, the clinician electronically signs, indicating acceptance of the information in the app, and the Physician Communication System uploads the information to the main data base, saving the information in the specific patient file for later retrieval.

In addition to allowing the clinician or another user to enter the patient files and modify the data, the Physician Communication System can automatically generate order forms according to the selected prosthetic device. Thus, after the clinician has electronically signed the patient's file, the Physician Communication System generates order forms for the specific prosthetic device chosen by the clinician and ads the order forms to the patient's file. The Physician Communication System can also be configured to send the order forms to an administrative person for ordering. This allows the Physician Communication System to automatically populate the order forms with the correct L-codes specifically identified for Medicare billing purposes.

After the new patient meets with and is evaluated by the clinician, the new patient must confirm the chosen prosthetic device with a physician. The Physician Communication System facilitates the process of the new patient's evaluation by the physician by simplifying the evaluation process with the use of prepopulated fields. Upon arrival at a physician's office, the new patient will sign in, check in, or otherwise notify the physician's office staff that the patient has arrived for the appointment. At this point, the office staff can open the app and access the Physician Communication System. In a preferred embodiment, the physician's office will have a dedicated smart device, usually and tablet, that is specifically used for accessing the Physician Communication System. The staff will access the app, entering a unique user identification and password, and thereby gain access to the Physician Communication System database. It is also contemplated that the unique identification and password will trigger data in the Physician Communication System related to the particular physician's office such as any subscription, fee structure, or other information particular to the specific physician's office.

When the physician's office staff signs into the Physician Communication System, it will automatically download any updated data or changed records necessary for the Physician Communication System application to run on the physician's smart device. This would include information related to updated L-codes, any software updates, etc. In addition, the physician's login and password will correspond to a list of patients maintained on the main Physician Communication System database. The Physician Communication System will recognize that the physician has signed in, and will download any updated information from the files on the physician's patients to the physician's smart device. Therefore any changes that the clinician made to one of the physician's patient's files will be automatically downloaded into the physician's smart device updating the files as necessary to ensure that the physician has the most current information. In the case of a new patient, the physician's office enters the patient's name in the app and the Physician Communication System downloads the patient's file to the physician's smart device, thereby adding that patient to the list of patients associated with the specific physician in the Physician Communication System database.

Once the new patient is signed in and the file is downloaded to the physician's smart device, the physician selects the new patient's file from a list of the physician's patients. It should be appreciated that in the event that a single patient has more than one prosthetic device, or needs more than one device, there will be an additional step for the physician to select the particular device to review.

When the physician enters the new patient's name into the smart device, the Physician Communication System software retrieves and displays the new patient's file information as a dynamic template. The Physician Communication System software pulls up all the information that the clinician entered as well as the information automatically populated by the software. The software contains various sections with patient specific information including a section that has recommendations of criteria that the clinician specified as determinative of the particular prosthetic selected for the patient. This will be what the physician reviews when deciding to accept or edit the recommendation of the clinician.

When the physician has the new patient's file uploaded on the smart device, the physician can begin the interview or examination of the new patient. As the physician is examining or interviewing the new patient, the physician is able to consult the Physician Communication System app and review the findings and recommendations of the clinician. The physician then evaluates the new patient to ensure that the patient meets the criteria listed in the Physician Communication System program for the particular device chosen.

Since the clinician is typically not a medical doctor, the physician must conduct an evaluation of various parameters and characteristics of the new patient to ensure that the new patient will be able to correctly use or benefit from the specific device selected by the clinician. Since there are multiple prosthetic devices available for the application, it is incumbent on the physician to ensure that the new patient has the physical capability, and the mental discipline, to fully use the prosthetic selected by the clinician. Thus, the physician's evaluation serves as a check on the clinician's recommendation of a specific prosthetic device.

The physician will check the new patient for various characteristics in order to ensure the proper and best prosthetic device is selected. The physician will check and evaluate things such as muscle strength of various muscles, desired activity levels, or other physical or mental requirements for use of the particular prosthetic that was selected by the clinician. If the physician decides that the patient is able to physically and mentally handle the prosthetic selected by the clinician, and agrees with the information contained in the Physician Communication System, the physician electronically signs the patient's file, selects a confirmation button, and the Physician Communication System software updates the new patient's file with the physician's approval.

Optionally the physician will have a choice on the Physician Communication System software to print a summary of the record for the physician's file. This will allow the physician to maintain a paper copy in the office. A physician may desire to have the paper copy for various reasons such as internal audits, or if a colleague needs to review the file but doesn't need access to the active Physician Communication System software.

If during the examination of the new patient, the physician finds an entry that needs to be changed, or finds an entry where further comments are necessary, the Physician Communication System is designed such that the physician can easily enter the updated or supplemental information. For example, if the physician determines that the new patient's quadriceps are not strong enough to fully utilize the selected prosthetic, the physician would enter this finding in the notes in the Physician Communication System. Thus, the dynamic nature of the Physician Communication System will allow the physician to change, delete, enter, move or otherwise manipulate the text in the various fields contained in the Physician Communication System. In a case where the physician determines that the new patient cannot meet the criteria for the selected device, the physician can enter into the Physician Communication System the corrected information. The Physician Communication System will then upload the edited information on the smart device and send it back to the main data base. From there, the Physician Communication System can send a notification to the clinician that the information for the new patient has been altered. The clinician can then download the information, review it for the changes made by the physician, and use the updated information entered by the physician to select an alternate prosthetic device based upon the comments by the physician.

The method of the instant invention uses similar steps when a patient is an existing patient and desires to get a new device. With this scenario, the existing patient will make an appointment with the clinician to discuss the present device and the possibility for obtaining a new device. When the patient meets with the clinician, the clinician uses the Physician Communication System and enters the existing patient's name to retrieve the patient specific file on smart device. The existing patient's file will already be downloaded to the smart device through the update feature of the Physician Communication System. Thus, when the existing patient arrives for the appointment, the clinician simply retrieves the patient's file from the smart device.

Once the existing patient and the clinician meet, the clinician with gather information from the existing patient regarding the patient's current device and the patient's desire or need for a new device. The clinician discusses the patient's desire for new device including the reasons a new device is necessary or desirable. The interview by the clinician covers any new activities the existing patient desires to participate in, and any significant changes in current activities that would require an alternate device. Based on this interview, the clinician verifies the physical attributes of the existing patient to ascertain what characteristics the new device will need to possess to satisfy the desires of the patient and to comply with the specific medical needs of the patient. The clinician also determines the K Level of the existing patient to see if it has changed since the last time the existing patient was treated or seen by the clinician or a physician. If the K Level of the patient has changed, the clinician will make an appropriate adjustment.

The clinician can also discuss with the existing patient any physical issues that are changed from previous evaluations and address any concerns the patient may have with the particular device currently in use. The Physician Communication System provides information blocks where the clinician notes the activities that the existing patient desires to participate in and evaluates the activities that are supported by various prosthetic devices. Once the clinician determines the proper components necessary for the new prosthetic device to have to accomplish the patient's goals, the clinician makes a selection of a new prosthetic device that best fits the desires and needs of the patient. The remaining steps in selecting the fields in the Physician Communication System are similar to the steps used with a new patient, including the automatic population of the various fields in the Physician Communication System.

pertinent to said physician.

1. A method for selection of a proper prosthetic device for a patient utilizing joint decision making and information gathering by a patient, clinician, and physician utilizing a computerized Physician Communication System, said method comprising:
Providing a Physician Computer System having a smart device in communication with a computer system storing a database containing patient specific information and prosthetic device specific information;
Providing software loaded on said smart device capable of inputting prosthetic device information and patient information, said software capable of uploading and downloading said information from said database in real time;
Having a clinician evaluate a patient to gather information from said patient, and enter said patient's information on said smart device, Having said clinician select a prosthetic device for treatment of said patient based upon said clinician's evaluation, and upload said selection to said PCS database through said smart device;
Having a physician consult said PCS database and evaluate said patient for the physical and mental factors necessary for use of said selected prosthetic device;
Having said physician verify that the physical and mental requirements for said prosthetic device selection are met by said patient, and having said physician accept said prosthetic device selection and input a verification of said selected prosthetic device into said database through said smart device.

2. A method for selection of a proper prosthetic device according to claim 1, further comprising:
Providing a Physician Computer System having a smart device in communication with a computer system storing a database containing patient specific information and prosthetic device specific information; wherein said information is dynamic and capable of being updated and manipulated in real time, and said smart device can selectively download and upload data to said database according to parameters selected by a user.

3. A method for selection of a proper prosthetic device according to claim 1 wherein said patient's information includes said patient's particular condition, goals, activities in which said patient desires to participate, treatment options, costs, risks, physical requirements, and wherein said prosthetic device information includes K codes, corresponding L-codes, any particular requirements for the specific device, and other device specific information necessary for the treatment of said patient and or evaluation and selection of a specific prosthetic device.

4. The method of claim 1 wherein said software comprises preloaded information related to said prosthetic devices organized into dynamic checkboxes wherein said clinician and said physician touch said checkbox to indicate that such information is applicable to said patient.

5. A method for prescribing a prosthetic device for a patient comprising:
Entering patient information into a smart device or computer installed with software capable of receiving, storing, and transmitting said information to a database, said database accessible to other devices through the Internet;
Sending said patient to a clinician for an initial recommendation of prosthetic device wherein said clinician enters patient information and or prosthetic device information into a smart device or computer that transmits said information to said database;
Evaluating said patient and said initial recommended prosthetic device for compatibility;
Determining whether said initial recommended prosthetic device is appropriate for said patient;
If appropriate, Verifying said initial recommended prosthetic device through said smart device wherein said smart device transmits said verification to said data base.

6. A method according to claim 5 further comprising;
Determining that said initial recommended prosthetic device is not appropriate for said patient;
Rejecting said initial recommended prosthetic device by deselection on said smart device;
Evaluating said patient further for alternate prosthetic device requirements; and
Inputting alternate recommendations or requirements for said patient into said smart device wherein said smart device transmits said alternate recommendations or requirements to said database for storage and retrieval.

7. A method according to claim 5 further comprising;
Converting said information entered by said clinician and sent to said database to corresponding information pertinent to said physician.
